(19) United States
(12) Patent Application Publication
(10) Pub. No.: US 2008/0082058 A1
(43) Pub. Date: Apr. 3, 2008

(54) Debriding Callus Foot Tissue

(76) Inventor: Gary S. Wallach, Coral Springs, FL (US)

Correspondence Address: ADVANTEDGE LAW GROUP, LLC, 3301 NORTH UNIVERSITY AVE., SUITE 200, PROVO, UT, 84604, US

(21) Appl. No.: 11/860,511

(22) Filed: Sep. 24, 2007

Related U.S. Application Data
(60) Provisional application No. 60/848,947, filed on Oct. 2, 2006.

(51) Int. Cl. A61M 35/00

(52) U.S. Cl. 604/293

(57) ABSTRACT

An exemplary process of debriding callus foot tissue is disclosed. The exemplary process includes applying a chemical peel to callus foot tissue and using instrumentation to remove the chemically treated callus foot tissue. In certain embodiment, the process includes preparing the chemical peel, including customizing at least one attribute of the chemical peel based on at least one attribute of the callus foot tissue.

### RELATED APPLICATIONS

The present application claims priority under 35 U.S.C. §119(e) to U.S. Provisional Patent Application No. 60/848,947 by Gary S. Wallach, filed on Oct. 2, 2006, and entitled “Removing Callus Tissue,” the contents of which are hereby incorporated by reference in their entirety.

### BACKGROUND INFORMATION

Debridement is the removal of dead, damaged, or infected tissue. In the field of podiatry, standard debridement techniques typically include surgical removal of dead, damaged or infected foot tissue, including callus foot tissue. Manual and powered instruments are commonly used for this purpose. For example, using standard podiatric techniques, a podiatrist may utilize a scalpel or other manual cutting tool to surgically cut or shave callus foot tissue, or a podiatrist may use a powered instrument such as a high-speed drill that spins a burr designed to surgically remove callus foot tissue.

However, conventional foot tissue debridement techniques have limitations. For example, simple, manual use of a scalpel to remove foot callus tissue is typically slow, painful, and prone to human error. Powered instruments can debride some foot tissue faster than manual instruments, but limitations remain. For example, great care must be used to avoid inflicting unnecessary pain, tearing tissue, and causing bleeding. In fact, certain burrs designed for abrasive debridement have been impractical, as well as unsuccessful in the market, namely because of their tendency to inflict pain, tear tissue, and cause bleeding. These limitations are especially problematic when working with thick, hardened, or fissured callus foot tissue. Accordingly, the burrs commonly available in the modern market are in general limited to those designed for less abrasive and consequently less efficient debridement of foot tissue. The use of such burrs can require significant treatment times, especially when used to debride thick and hardened callus tissue. Moreover, such burrs tend to produce excessive amounts of fine particle dust when used on callus foot tissue.

The results produced by standard debridement techniques also leave room for improvement. For example, conventional surgical debridement of callus foot tissue may achieve a certain level of success in removing unwanted tissue, but success is limited by the nature and difficulty of working with (e.g., cutting, grinding, shaving, sanding, etc.) hardened callus foot tissue. Moreover, callus foot tissue that has been removed using standard debridement techniques often reforms at an unacceptable rate.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, like reference numbers designate like elements.

FIG. 1 is a flowchart of an exemplary method of debriding callus foot tissue.

FIG. 2 illustrates a pre-treatment state of a foot having callus tissue.

FIG. 3 illustrates a post-treatment state of the foot of FIG. 2.

FIG. 4 illustrates a pre-treatment state of a heel of a foot having callus tissue.

FIG. 5 illustrates a post-treatment state of the heel of FIG. 4.

FIG. 6 illustrates a pre-treatment state of a foot having metatarsal callus tissue.

FIG. 7 illustrates a post-treatment state of the foot of FIG. 6.

FIG. 8 illustrates a pre-treatment state of a foot having scaling tissue.

FIG. 9 illustrates a post-treatment state of the foot of FIG. 8.

### DETAILED DESCRIPTION

Exemplary embodiments of podiatric debridement are described herein. The embodiments are illustrative rather than restrictive. The embodiments described herein are described in relation to debridement of “callus foot tissue,” which term, as used herein, may include, but is not limited to, hardened foot tissue, excessively thick and hardened foot tissue, foot tissue having bleeding lesions, foot tissue having fissures, scaling tissue, corns, tyloma, hyperkeratosis, other thickened, devitalized, or significantly keratinized tissue such as plaques or scales, and any other foot tissue where reduction of the tissue would be efficacious and beneficial, including beneficial reduction before initiation or application of other modalities, medications, or medical procedures.

The examples described herein have been found to generally debride callus foot tissue in an efficient, effective, and substantially pain-free manner. The exemplary processes described herein can reduce callus foot tissue remarkably more thoroughly, effectively, and efficiently than conventional debridement techniques. Thus, callus foot tissue can be efficiently removed or reduced to a degree that cannot be achieved by standard debridement techniques.

Moreover, the processes described herein typically reduce patient discomfort or pain as compared to standard debridement techniques. For example, the exemplary processes facilitate avoidance of, or at least significant and remarkable decreases in, instances of discomfort, pain, tissue tearing, and bleeding that may otherwise occur when standard debridement techniques are used.

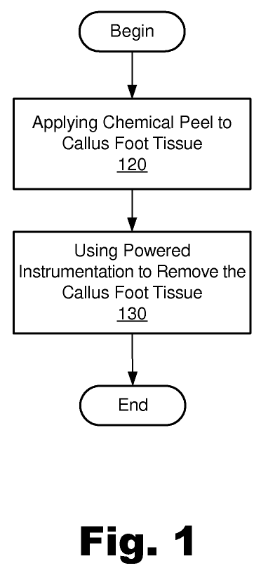

FIG. 1 is a flowchart illustrating an exemplary method of debriding callus foot tissue. In certain applications, the method may be performed by a licensed professional or under supervision of a licensed profession such as a podiatrist. While FIG. 1 illustrates exemplary acts according to one embodiment, other embodiments may add to and/or modify any of the acts shown in FIG. 1.

In step 120 of FIG. 1, a chemical peel is applied to callus foot tissue. The chemical peel may be configured and used to chemically soften the callus foot tissue and may include one or more tissue debriding, softening, and/or desquamating agents capable of softening callus foot tissue. The chemical peel may include any suitable chemical composition that when applied will generally soften callus foot tissue, preferably in an efficient, safe, and controllable manner.

It will be appreciated that chemical treatment of callus foot tissue presents different considerations and challenges than chemical treatment of tissue on other parts of the body. On a histological level, a human foot includes more functional layers than other parts of the human anatomy. Moreover, callus foot tissue is often thicker and harder than callus tissue typically found on other parts of the body. Accordingly, the chemical peel used in step 120 is configured specifically for softening callus foot tissue in an efficient manner. For example, one or more agents in the chemical peel should have sufficient strength, potency, and/or concentration to efficiently effect satisfactory chemical penetration and/or softening on callus foot tissue.

Application of the chemical peel may significantly soften the callus foot tissue. Such chemical softening of callus foot tissue is markedly different from merely moisturizing or hydrating skin using standard moisturizing or softening creams such as urea creams. Such creams cannot efficiently soften callus foot tissue in a manner that is acceptable for the exemplary methods disclosed herein. Moreover, the use of such standard creams typically requires a lengthy treatment period lasting days or weeks, as well as active patient compliance over the length of the treatment.

The chemical peel may include, but is not limited to, an alpha-beta peel, an alpha-beta peel mixture, and a chemical composition including one or more tissue softening agents configured to suitably soften callus foot tissue. Softening agents may include but are not limited to retinoic acid (“Retin-A”), lactic acid, glycolic acid, salicylic acid, alpha hydroxy acid (“AHA”), beta hydroxy acid (“BHA”), other acids capable of suitably softening callus foot tissue for the methods disclosed herein, and any suitable combination of such agents. In certain embodiments, the chemical peel may include desquamating agents such as salicylic acid.

The chemical peel may include one or more vehicular and/or penetrating agents such as dimethyl sulfoxide (“DMSO”) for carrying the active ingredients (i.e., debriding agents) and/or penetrating the callus foot tissue. The chemical peel may include or be in the form of a surfactant with which any of the above-listed agents may be used.

Although not shown in FIG. 1, an exemplary process may include preparing the chemical peel. Preparation of the chemical peel may be said to be an act that is performed separate from or as part of step 120. Preparation of the chemical peel may include mixing together one or more ingredients such as tissue softening agents (e.g., alpha and beta acids) and vehicular and/or penetrating agents to form the chemical peel. For certain chemical peels to be effective, preparation of the peels should be performed just prior to their application rather than preparing the peels in advance.

Preparation of a chemical peel may include customizing the chemical peel (at least one attribute of the chemical peel) based on one or more factors, including, but not limited to, the treatment to be performed, patient preferences, and patient attributes (e.g., skin type, skin color, tissue hardness, tissue thickness, tissue location, tissue condition, etc.). In certain embodiments, for example, at least one attribute of the chemical peel is customized based on at least one attribute of the callus foot tissue (e.g., hardness, thickness, or location of the callus tissue). Attributes of the chemical peel that may be customized include, but are not limited to, the ingredients used in the chemical peel and the amount, concentration, and/or type of various ingredients such as softening and penetrating agents.

In certain embodiments, at least one attribute of the chemical peel may be adjusted based on a Fitzpatrick skin type score for a patient. Fitzpatrick skin type scores are well known. As an example, a higher concentration of an active ingredient (e.g., a tissue softening agent such as salicylic acid) may be used in a chemical peel to be applied to a skin type having a higher Fitzpatrick skin type score, while a lower concentration may be used for a skin type having a lower Fitzpatrick skin type score.

Other examples of customizing the chemical peel may include using stronger acids or concentrations of acids or other agents for softening especially thick or hard callus tissue, or using a higher concentration of penetrating agent such as DMSO to penetrate especially thick or hard callus tissue. In other words, the ingredients of the chemical peel may be adjusted as may best suit a particular treatment and/or skin condition (i.e., best debride or penetrate particular callus foot tissue).

Where a chemical peel is a mixture of ingredients, the ingredients may be combined together in any suitable manner, including using manual, automatic, or a combination of manual and automatic steps.

Chemical peels including salicylic acid in a range between approximately 50% and 70% in DMSO (5 mL), i.e., 500-700 mg of salicylic acid per one mL of DMSO, have been found to effectively and efficiently soften callus foot tissue. In certain examples, the concentration of the salicylic acid in DMSO may be adjusted between approximately 70% and 50% in DMSO (or other vehicular agent) to customize a chemical peel for a particular treatment, patient, skin type, etc. As mentioned, the customization may be based on one or more treatment-specific parameters such as the Fitzpatrick skin type score of a patient. Chemical peels including a mixture of approximately 20% glycolic acid in solution (5 mL), i.e., 200 mg for one mL of solution, have also been found to effectively and efficiently soften callus foot tissue.

A chemical peel may be applied to callus foot tissue in any suitable manner and using any potentially helpful tools. For example, one or more cotton swabs and/or cotton buds may be used to apply the chemical peel to callus foot tissue.

Step 120 may include applying the chemical peel to one or more locations of a foot, including but not limited to a heel, ball, metatarsal area, sides, and toes of the foot. In certain embodiments, step 120 may include selectively applying the chemical peel to certain locations of a foot while avoiding application of the chemical peel to other locations on the foot. In certain examples, step 120 may include applying the chemical peel to callus foot tissue while avoiding application of the chemical peel to potentially sensitive areas of the foot such as on or near skin between toes or adjacent to the web space on the plantar surface of the foot.

The chemical peel should be applied for a suitable period of time that allows for penetration of the chemical peel (or at least the active ingredients of the chemical peel) into and sufficient softening of the keratin of the callus foot tissue. In certain embodiments, applying the chemical peel in step 120 includes allowing sufficient time for the chemical peel to significantly soften the callus foot tissue. It has been found that waiting approximately four to five minutes generally allows for effective chemical penetration and softening of callus foot tissue. In certain embodiments, the waiting time is limited to a maximum of approximately four to five minutes.

In certain embodiments, the length of the waiting time for the chemical peel to soften tissue may be adjusted on a treatment-by-treatment basis, based on certain factors such as the thickness, hardness, and location of callus foot tissue. For example, the chemical peel may be allowed to soften tissue for a shorter period of time for callus tissue having a minimal thickness, or the waiting time may be extended for thicker or harder callus tissue.

In certain embodiments, step 120 includes actively drying the chemical peel, or at least the top layer of the chemical peel, on the callus foot tissue. This may be accomplished using an electrically-powered fan. Other drying techniques and tools may be used, including applying heat (e.g., using a heat lamp or blow dryer) to dry the chemical peel. Actively drying the chemical peel can help speed up the treatment process.

Performance of step 120 prepares callus foot tissue for effective and efficient debridement using instrumentation. In general, the callus foot tissue is significantly softened such that powered instrumentation can be used to remove the chemically debrided callus foot tissue efficiently, effectively, and in a generally pain-free manner. Typically, the results are remarkably improved as compared to results produced by standard podiatric debridement techniques.

In step 130, instrumentation is used to remove the chemically softened callus foot tissue. Instrumentation may include any manual, powered, or combination of manual and powered instrumentation capable of surgically or physically debriding the chemically treated callus foot tissue.

In certain embodiments, a powered instrument producing a generally circular motion is used. For example, a powered drill with one or more cutting or buffing bits (i.e., burrs) may be used. The powered instrumentation may include a “Jan L” rotary powered nail drill with an RPM rating of approximately 25,000 to 30,000 with a dust extractor, or any powered instrument or system capable of producing powered motion of a disc, file, or other burr. For example, the powered instrument may include any powered drill that can accept the mandril of a suitable bit for debriding callus foot tissue. Suitable powered instruments developed in the future may also be used to drive a disc, file, drum, bit, or other burr useful for debriding callus foot tissue that has been softened by a chemical peel.

The powered drill may support using different bits. For example, a course bit such as a Cherokee circular disc (or other rotary flat-headed file) with multidirectional cutting teeth may be used to debride very thick and/or hardened callus tissue, and a less course bit such as a diamond circular burr (e.g., an “Umbrella” burr) may be used for callus foot tissue that is not as thick or hardened. The Cherokee circular disc may be used to remove thicker callus foot tissue. If the callus foot tissue is not very thick to begin with, or once the Cherokee circular disc has been used to buff the thick callus foot tissue, the diamond burr may be used to more gently buff the callus foot tissue. By supporting the use of variable bits, the powered instrumentation enables different degrees of powered cutting and/or buffing operations. Accordingly, step 130 may include sub-steps for different degrees and/or types of debriding.

By first softening callus foot tissue with a chemical peel in step 120, more aggressive (e.g., abrasive) tools can be used in step 130 than can practicably be used in standard surgical debridement techniques. For example, the Cherokee burr mentioned above has been a market failure at least because its abrasive qualities have been known to cause pain, tearing, and bleeding when used in standard surgical debridement techniques. Thus, this burr has been labeled as impractical for use in standard surgical debridement techniques. Remarkably, however, the burr has been found to perform efficiently, effectively, and in a substantially pain-free manner when used in the process of FIG. 1. Significantly, the chemical peel applied in step 120 efficiently softens callus foot tissue such that the Cherokee burr can be used in step 130 generally without inflicting pain, tearing skin, or causing bleeding.

In alternative embodiments, other suitable powered instrumentation may be used. Alternative or in addition to a circular debridement surface, other types of surfaces and/or motions may be employed by powered instrumentation to debride the callus foot tissue. For example, a rotating cylinder with a modified drill bit (e.g., a sanding drum with teeth) could be used. By way of another example, an oscillating bit such as a saw blade that moves forward and back could be used. Yet another example includes using a powered instrument that cuts in and out in the sagital plane (or up and down if the powered instrument is held on its side). Cross-cutting instrumentation may also be used.

In step 130, the depth, speed, and area of removal of the callus foot tissue may be determined by the experience of the operator using or directing the use of the instrumentation.

Steps 120 and 130 may be repeated for different layers of callus foot tissue, thereby allowing layer-by-layer removal of the callus foot tissue. For example, after steps 120 and 130 have been performed to remove one or more first layers of callus foot tissue, the steps may be repeated for one or more other layers (sub-layers) of the callus foot tissue. This is especially useful for treating very thick and hardened callus foot tissue as is commonly found on the bottom of human feet.

Other treatment options may be used to enhance the process of FIG. 1. For example, pre-treatment of callus foot tissue using a standard podiatric debridement technique such as manual scalpel debridement may be used. By way of another example, a standard debridement technique may be used on callus foot tissue found at certain locations on a foot, including callus foot tissue on the dorsum of a toe, for example.

By using powered instrumentation to debride callus foot tissue after the tissue has been chemically treated in step 120, a mini-scale, high speed debridement (e.g., cutting and/or buffing) of chemically treated callus foot tissue is performed. The mini-scale debridement in combination with the chemical treatment is able to remove callus foot tissue, without tearing at fissures of the callus foot tissue, which is a common problem of standard surgical debridement techniques. The combination of chemical treatment and powered debridement generally produces an efficient and pain-free process, in contrast to conventional debridement techniques.

It has been found that many treatments using the process of FIG. 1 can be performed in less than twenty minutes for both feet, without patients experiencing the human errors, pain, and discomfort that are commonly associated with standard debridement techniques.

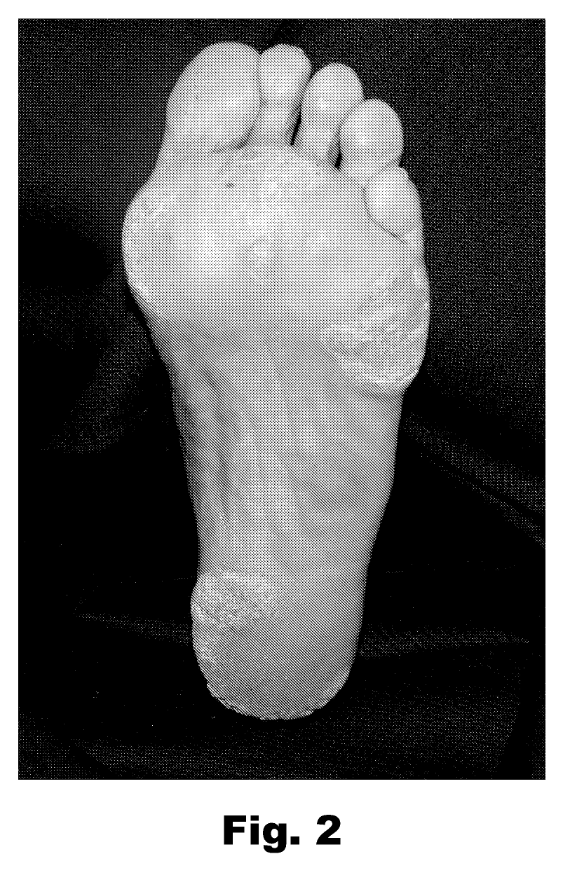

Typically, the results produced by performance of the process of FIG. 1 are remarkably improved as compared to results produced by standard podiatric debridement techniques. FIGS. 2-9 include show various human feet at pre-treatment and post-treatment states. The Figures depicting post-treatment states of feet show the feet as they existed after being treated using exemplary processes disclosed herein.

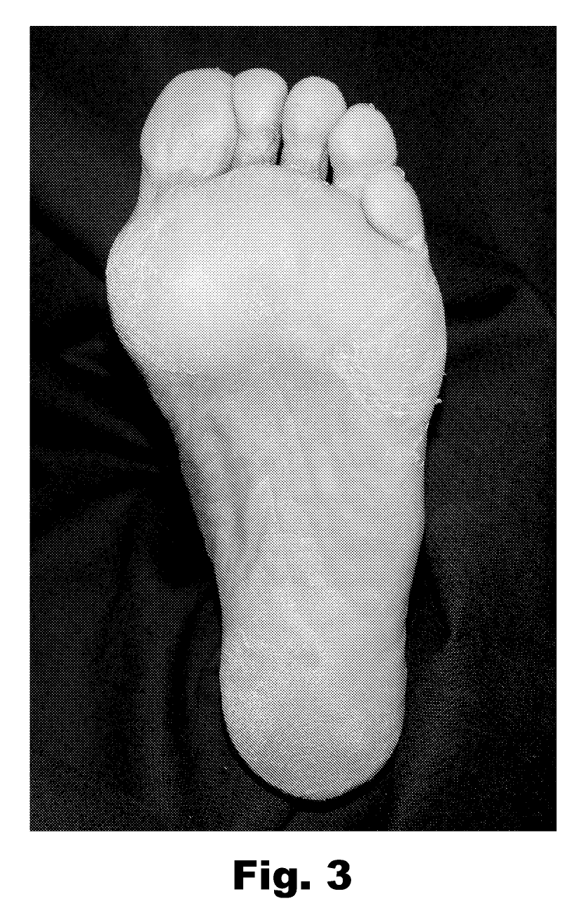

FIGS. 2 and 3 respectively illustrate pre-treatment and post-treatment states of a human foot. The callus foot tissue shown in FIG. 2 was treated using exemplary processes disclosed herein to produce the results shown in FIG. 3. As shown, the callus foot tissue in FIG. 2 has been reduced significantly.

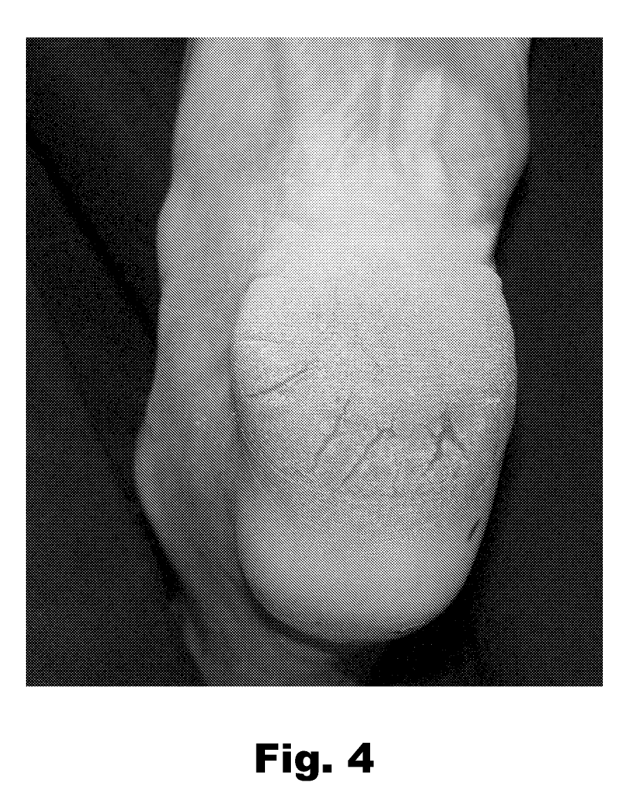

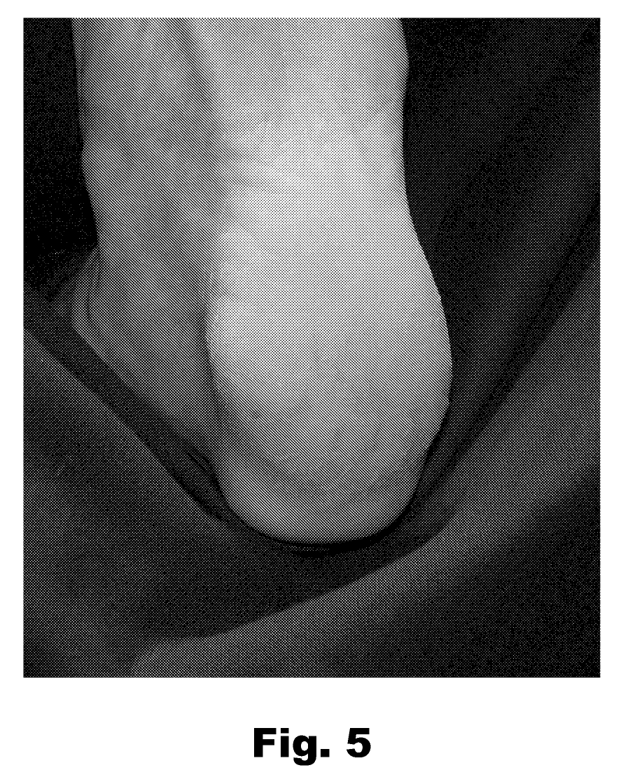

FIGS. 4 and 5 respectively illustrate pre-treatment and post-treatment states of a heel of another human foot. The callus foot tissue shown in FIG. 4 was treated using exemplary processes disclosed herein to produce the results shown in FIG. 5. As shown, the callus foot tissue in FIG. 4 has been reduced significantly.

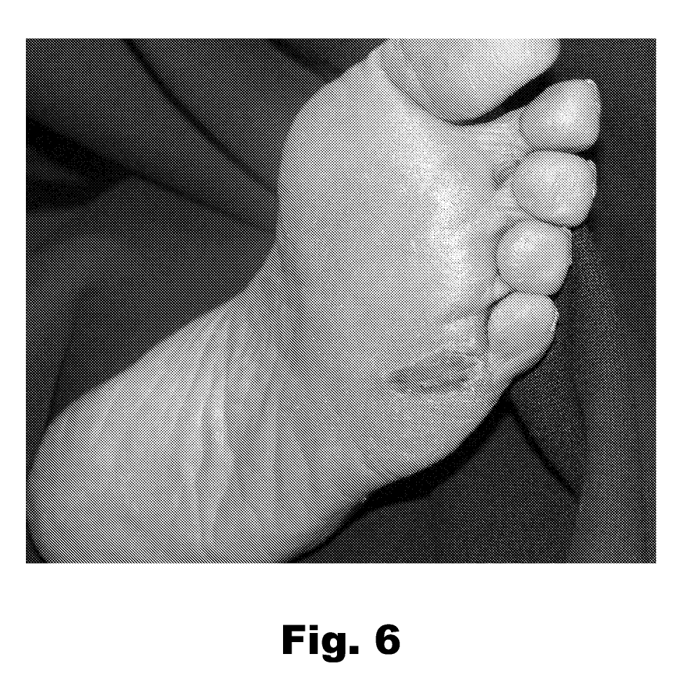

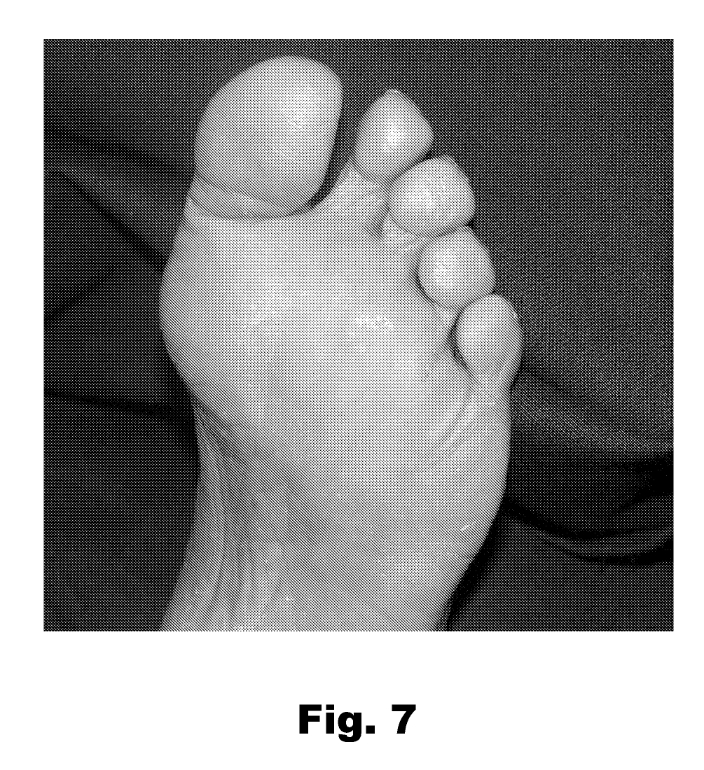

FIGS. 6 and 7 respectively illustrate pre-treatment and post-treatment states of another human foot having metatarsal callus tissue. The callus foot tissue shown in FIG. 6 was treated using exemplary processes disclosed herein to produce the results shown in FIG. 7. As shown, the metatarsal callus foot tissue in FIG. 6 has been reduced significantly.

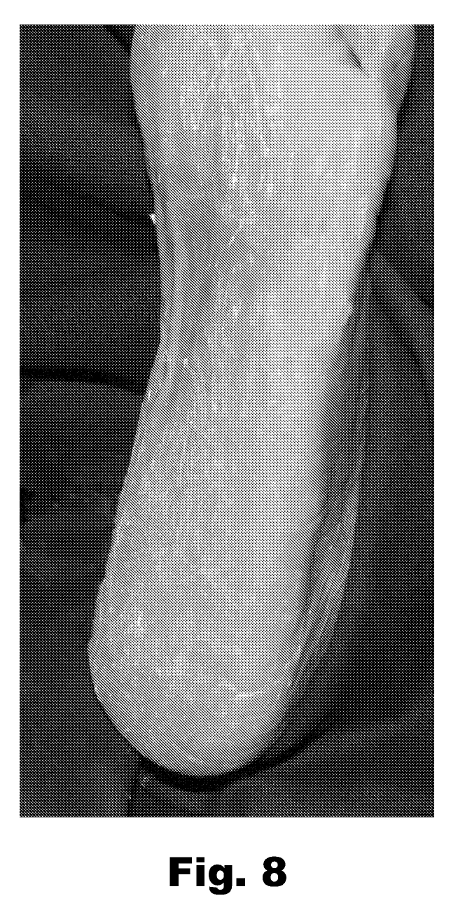

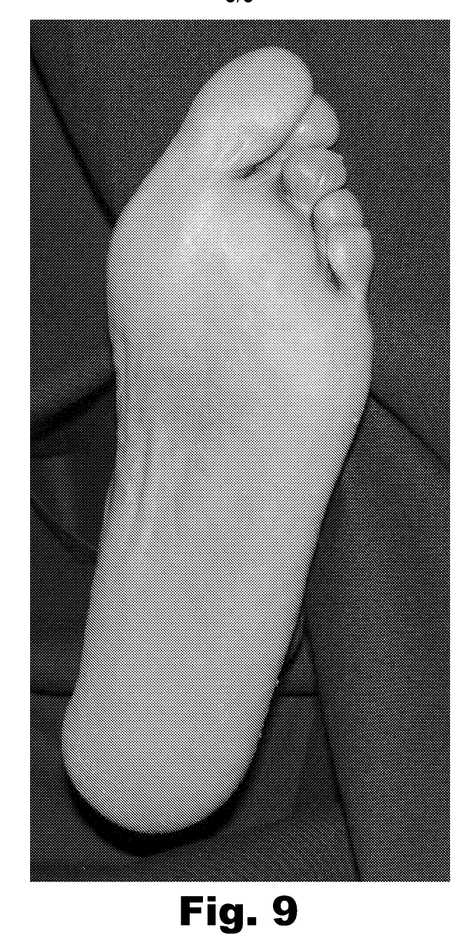

FIGS. 8 and 9 respectively illustrate pre-treatment and post-treatment states of yet another human foot having scaling tissue. The scaling foot tissue shown in FIG. 8 was treated using exemplary processes disclosed herein to produce the results shown in FIG. 9. As shown, the scaling foot tissue in FIG. 8 has been reduced significantly.

Tables 1-4 below include data representative of observed results produced by performance of the podiatric debridement processes described herein. Table 1 includes data for treatment of callus tissue on the heels of feet, Table 2 includes data for treatment of callus tissue on the balls of feet, Table 3 includes data for treatment of callus tissue on the toes of feet, and Table 4 includes data for treatment of callus tissue on the marginal areas of feet.

As used herein for purposes of explaining the results produced by the practice of processes described herein, as represented by the data in Tables 1-4, the “heel” of a foot refers to the region of the foot formed by the calcaneus and that generally contacts the ground or footwear worn on the foot, the “ball” of a foot refers to the metatarsophalangeal area of the foot and the metatarsal arch, the “toes” of a foot refer to the plantar surfaces of the digits, the medial side of the hallux and the lateral side of the fifth toe, and the “margin” of a foot refers to the lateral side of the foot from heel to metatarsophalangeal joint level.

In the results data of Tables 1-4, callus foot tissues have been categorized into four types based on attributes of the callus tissues. A podiatrist categorized the various callus foot tissues based on observation of the tissues. The four categories are: (1) excessive, thick callus tissue; (2) underlying fissure callus tissue; (3) bleeding lesion callus tissue; and (4) scaling skin callus tissue. The data for any of these categories included in a Table are also totaled in the Table.

The data in the Tables is representative of results produced by using the podiatric debridement processes disclosed herein to treat over one hundred different patients. The data is based on and representative of observations made by a podiatrist during post-treatment follow-up visits conducted approximately eight weeks after treatments were performed. The data in the Tables generally indicates an approximate percentage by which callus foot tissue has been reduced from its original condition. Thus, a “percent reduction” refers to the percentage by which callus foot tissue been reduced at approximately eight weeks after treatment, as compared to the original, pre-treatment state of the tissue. For example, a 75% reduction indicates that callus foot tissue at eight weeks post-treatment has been reduced by 75% as compared to its pre-treatment state.

In the Tables, the percentage reduction has been grouped into three different categories: “10% reduction,” meaning approximately 10%-49% reduction; “50% reduction,” meaning approximately 50%-74% reduction; and “75% reduction,” meaning 75%-100% reduction.

TABLE 1

| Evaluation Heel: | | | |
|---|---|---|---|
| Type of Lesion | 10% Reduction | 50% Reduction | 75% Reduction |
| (1) Excessive, thick | 28% | 47% | 25% |
| callus tissue | | | |
| (2) Underlying fissure | 10% | 65% | 25% |
| (3) Bleeding lesion | 75% | 0% | 25% |
| Total: % for all heel | 25% | 50% | 25% |
| lesion types | | | |

Turning now to the Tables, Table 1 includes data related to the treatment of the heels of human feet. As shown in Table 1, for treatments of excessive, thick callus tissue on the heels of feet, 28% of the treatments produced a “10% reduction,” 47% of the treatments produced a “50% reduction,” and 25% of the treatments produced a “75% reduction.” For treatments of underlying fissure callus tissue on the heels of feet, 10% of the treatments produced a “10% reduction,” 65% of the treatments produced a “50% reduction,” and 25% of the treatments produced a “75% reduction.” For treatments of bleeding lesion callus tissue on the heels of feet, 75% of the treatments produced a “10% reduction,” 0% of the treatments produced a “50% reduction,” and 25% of the treatments produced a “75% reduction.” For all treated categories of callus heel tissue, 25% of the treatments produced a “10% reduction,” 50% of the treatments produced a “50% reduction,” and 25% of the treatments produced a “75% reduction.”

TABLE 2

| Evaluation Ball: | | | |
|---|---|---|---|
| Type of Lesion | 10% Reduction | 50% Reduction | 75% Reduction |
| (1) Excessive, thick | 23% | 45% | 32% |
| callus tissue | | | |
| (2) Underlying fissure | 33% | 0% | 67% |
| Total: % for all ball | 23% | 43% | 34% |
| lesion types | | | |

Table 2 includes data related to the treatment of the balls of human feet. As shown in Table 2, for treatments of excessive, thick callus tissue on the balls of feet, 23% of the treatments produced a “10% reduction,” 45% of the treatments produced a “50% reduction,” and 32% of the treatments produced a “75% reduction.” For treatments of underlying fissure callus tissue on the heels of feet, 33% of the treatments produced a “10% reduction,” 0% of the treatments produced a “50% reduction,” and 67% of the treatments produced a “75% reduction.” For all treated categories of callus tissue on the balls of feet, 23% of the treatments produced a “10% reduction,” 43% of the treatments produced a “50% reduction,” and 34% of the treatments produced a “75% reduction.”

TABLE 3

| Evaluation Toes: | | | |
|---|---|---|---|
| Type of Lesion | 10% Reduction | 50% Reduction | 75% Reduction |
| (1) Excessive, thick | 26% | 44% | 29% |
| callus tissue | | | |
| Total: % for all toes | 26% | 44% | 29% |
| lesion types | | | |

Table 3 includes data related to the treatment of the toes of human feet. As shown in Table 3, for treatments of excessive, thick callus tissue on the toes of feet, 26% of the treatments produced a “10% reduction,” 44% of the treatments produced a “50% reduction,” and 29% of the treatments produced a “75% reduction.” For all treated categories of callus tissue on the toes of feet, which includes only excessive, thick callus tissue in this set of data, the numbers are the same.

TABLE 4

| Eval. Margins: | | | 75% |
|---|---|---|---|
| Type of Lesion | 10% Reduction | 50% Reduction | Reduction |
| (1) Scaling skin | 20% | 80% | 0% |
| callus tissue | | | |
| Total: % for all margins | 20% | 80% | 0% |
| lesion types | | | |

Table 4 includes data related to the treatment of the margins of human feet. As shown in Table 4, for treatments of scaling callus tissue on the margins of feet, 20% of the treatments produced a “10% reduction,” 80% of the treatments produced a “50% reduction,” and 0% of the treatments produced a “75% reduction.” For all treated categories of callus tissue on the margins of feet, which includes only scaling callus tissue in this set of data, the numbers are the same.

Treatments of callus foot tissue using the processes disclosed herein produced remarkably improved results as compared to results typically produced by standard debridement techniques. In addition to efficiently and effectively debriding callus foot tissue in a generally pain-free manner, the data in the Tables shows that reoccurrences of debrided callus foot tissue are improved when compared to the results produced by standard debridement techniques. It has been observed by a podiatrist that callus foot tissue that has been debrided as described herein tends to reform after treatment but at reduced rates as compared with the return of callus foot tissue that has been treated using standard techniques.

In certain implementations, instruction for performing the above-described processes may be provided. Such instruction may include any direction for preparing a chemical peel configured to suitably soften callus foot tissue, applying the chemical peel to the callus foot tissue, using instrumentation to remove the chemically softened callus foot tissue, and performing any of the other acts described herein. The instruction may be provided in the form of in-person training, pre-recorded media (e.g., audio and/or visual instruction), print materials, seminars, classroom instruction, and any other suitable form. Along with instruction for performing the above-described podiatric debridement processes, at least one ingredient of the chemical peel and/or at least one component of the instrumentation (e.g., a burr) may be provided, thereby facilitating performance of the processes.

The preceding description has been presented only to illustrate and describe exemplary embodiments with reference to the accompanying drawings. It will, however, be evident that various modifications and changes may be made thereto, and additional embodiments may be implemented, without departing from the scope of the invention as set forth in the claims that follow. The above description and accompanying drawings are accordingly to be regarded in an illustrative rather than a restrictive sense.

What is claimed is:

1. A method comprising:
applying a chemical peel to callus foot tissue, said chemical peel being configured to soften the callus foot tissue; and
using instrumentation to remove the chemically softened callus foot tissue.

2. The method of claim 1, said chemical peel comprising a tissue softening agent and a tissue penetrating agent.

3. The method of claim 2, said tissue softening agent including at least one of retinoic acid (“Retin-A”), lactic acid, glycolic acid, salicylic acid, alpha hydroxy acid (“AHA”), beta hydroxy acid (“BHA”).

4. The method of claim 1, said chemical peel comprising an alpha-beta peel.

5. The method of claim 1, said chemical peel including a concentration comprising salicylic acid and dimethyl sulfoxide (“DMSO”).

6. The method of claim 5, said concentration including a range of approximately salicylic acid 50% to 70% in DMSO.

7. The method of claim 1, said chemical peel including a mixture of approximately glycolic acid 20% in solution.

8. The method of claim 1, further comprising preparing said chemical peel, including customizing said chemical peel based on at least one of patient skin type, hardness of the callus foot tissue, thickness of the callus foot tissue, and location of the callus foot tissue.

9. The method of claim 1, said customizing including customizing a concentration of at least one of a tissue softening agent and a tissue penetrating agent included in said chemical peel.

10. The method of claim 1, wherein said applying includes actively drying said chemical peel.

11. The method of claim 1, wherein said applying includes allowing sufficient time for said chemical peel to significantly soften the callus foot tissue.

12. The method of claim 1, wherein said using said instrumentation includes using powered instrumentation to remove the chemically softened callus foot tissue.

13. The method of claim 12, said powered instrumentation including a power drill with an RPM rating of approximately 25,000 to 30,000 with a rotary flat-headed burr having multi-directional teeth.

14. The method of claim 1, said powered instrumentation including a power drill with a diamond burr.

15. The method of claim 1, wherein said applying and said using acts debride one or more first layers of the callus foot tissue.

16. The method of claim 15, further comprising repeating said applying and said using acts to debride one or more sub-layers of the callus foot tissue.

17. A method of debriding callus foot tissue, the method comprising:
preparing a chemical peel, including customizing at least one attribute of said chemical peel based on at least one attribute of the callus foot tissue;
applying said chemical peel to the callus foot tissue, said chemical peel including at least one tissue softeing agent configured to soften the callus foot tissue, said applying including allowing sufficient time for said chemical peel to significantly soften the callus foot tissue; and
using powered instrumentation and removing the chemically softened callus foot tissue.

18. The method of claim 17, said preparing including customizing said at least one attribute of said chemical peel based on a skin type attribute of a patient.

19. A method comprising:
providing instruction for debriding callus foot tissue, including instruction for
preparing a chemical peel configured to soften the callus foot tissue,
applying said chemical peel to the callus foot tissue, and
using instrumentation to remove the chemically softened callus foot tissue.

20. The method of claim 19, further comprising providing at least one of the following:
at least one ingredient for inclusion is said chemical peel, and
at least one component of said instrumentation.
